(19) United States
(12) Patent Application Publication
(10) Pub. No.: US 2024/0065997 A1
(43) Pub. Date: Feb. 29, 2024

(54) Methods and Compositions for Improving Insulin Production and Secretion

(71) Applicant: ABBOTT LABORATORIES, Abbott Park, IL (US)

(72) Inventors: José María LÓPEZ PEDROSA, Cenes de la Vega (ES); Ricardo RUEDA CABRERA, Granada (ES); Manuel MANZANO, Granada (ES)

(21) Appl. No.: 18/552,726

(22) PCT Filed: Mar. 31, 2022
(86) PCT No.: PCT/US2022/022712

§ 371 Date: Sep. 27, 2023

(30) Foreign Application Priority Data
Mar. 31, 2021 (EP) 21382265.3

(51) Int. Cl. A61K 31/198; A61K 31/047; A61K 47/26; A61K 47/42; A61K 47/44; A61P 3/10

(52) U.S. Cl. CPC A61K 31/198; A61K 31/047; A61K 47/26; A61K 47/42; A61K 47/44; A61P 3/10

(57) ABSTRACT

A method of improving insulin production in a subject in need thereof comprises administering a nutritional composition comprising lysine, arginine, and beta-hydroxy-beta-methylbutyrate (HMB) to the subject. A method of improving insulin secretion in a subject in need thereof comprises administering a nutritional composition comprising lysine, arginine, and HMB to the subject. A nutritional composition comprises about 0.01 to about 15 wt % HMB, about 0.03 to about 40 wt % lysine, and about 0.02 to about wt % arginine.

### FIELD OF THE INVENTION

The present invention relates to methods of improving insulin production in a subject, to methods of improving insulin secretion in a subject, and to nutritional compositions which employ lysine, arginine, and beta-hydroxy-beta-methylbutyrate (HMB).

### BACKGROUND OF THE INVENTION

Pancreatic β-cells produce and secrete insulin, which is the hormone that regulates blood glucose concentration. β-cells must be able to produce, store, and secrete insulin in sufficient concentrations to maintain normal levels of glucose in the blood, i.e., euglycemia. Physiologically, euglycemia is governed by the balance between peripheral insulin sensitivity (how readily body cells in the periphery tissue, such as muscle and fat, can absorb glucose) and insulin secretion: when insulin sensitivity is reduced, insulin secretion is increased. Since β-cells are very sensitive to blood glucose concentrations, changes in the body's ability to maintain equilibrium in blood glucose concentration greatly influences their function. When elevated glucose levels and insulin production are prolonged, the pancreas loses the ability to adapt β-cell mass to insulin demand over time, thereby leading to a decrease in functional β-cell mass. Dysregulation of β-cell functional mass represents a key mechanistic factor linked to the onset and progression of diabetes.

Although there has been continuous progress in treating diabetes through pharmaceutical therapy, handling diabetes progression is a critical issue because the etiology and mechanisms of diabetes development are still not fully understood. Two principal problems exist: how to cure diabetes and how to lower the prevalence of diabetes by primary and secondary prevention.

Insulin resistance and β-cell dysfunction have important roles in the pathogenesis and evolution of diabetes. Insulin resistance, often present years before diabetes is diagnosed, reflects a diminished response to insulin it its key target issues, such as muscle, liver and adipose tissue, and has been shown to predict the development of the disease. β-cell function is already reduced in subjects with impaired glucose tolerance, such as patients diagnosed with prediabetes, and is even more reduced in subjects with type 2 diabetes. According to a UKPDS study, β-cell function in subjects with type 2 diabetes might be reduced by 50% at diagnosis with a 5% decline per each subsequent year, suggesting the beginning of deterioration several years before disease onset. Lancet, 352 (1998): pp. 837-853 and pp 854-865; Br Med J, 317 (1998), pp. 703-713 and pp. 713-720.

Since the deficit of β-cell functional mass is a necessary and early condition for the development of type 2 diabetes, methods providing for β-cell restoration and/or β-cell regeneration and preserving functional islet integrity are desirable for type 2 diabetes prevention, delay of progression, treatment, remission, and potentially a cure.

Currently, the use of antidiabetic medication is widespread. Available pharmaceutical therapies for treating diabetes have been developed as “symptomatic” medications since they primarily act to reduce elevated blood glucose levels. However, it has been described that monotherapy with antidiabetic medications (e.g., metformin, rosiglitazone, and glyburide) fails over time, albeit with differences in the rates of decline. In addition, current therapies do not completely abolish the progressive loss of β-cell function, and their use is also associated with hypoglycemia and weight gain. In order to be capable of preventing the onset and progression of type 2 diabetes, treatment should also stop β-cell dysfunction and promote the restoration of fully functional β-cell mass, independently of reducing hyperglycemia. Management of type 2 diabetes should ideally involve early and simultaneous treatment of insulin resistance and β-cell dysfunction.

In addition to pharmaceutical therapies, as mentioned above, major effort has been made in understanding the effective preventative management of both prediabetes and diabetes through exercise and nutritional intervention. Preventative strategies for the management of diabetes progression, for example the progression from prediabetes to diabetes, or the progression from gestational diabetes to type 2 diabetes following pregnancy, should focus on improving insulin secretion and action, minimizing the factors associated with diabetes, especially insulin resistance, preventing hyperglycemia, and reestablishing muscle structure and functions.

Accordingly, methods of improving insulin secretion and production are desirable, particularly for patients suffering from diabetes or prediabetes. It is also desirable to provide methods of improving insulin secretion and production that help to delay progression of, prevent or reverse type 2 diabetes or prediabetes. A nutritional intervention that can help address the above limitations associated with existing diabetes treatments is also desirable.

### SUMMARY OF THE INVENTION

In one embodiment, the invention is directed to a method of improving insulin production in a subject in need thereof, comprising administering a nutritional composition comprising lysine, arginine, and beta-hydroxy-beta-methylbutyrate (HMB) to the subject.

In an additional embodiment, the present invention is directed to a method of improving insulin secretion in a subject in need thereof, comprising administering a nutritional composition comprising lysine, arginine, and HMB to the subject.

In a further additional embodiment, the present invention is directed to a nutritional composition comprising about 0.01 to about 15 wt % of HMB, about 0.03 to about 40 wt % of lysine, and about 0.02 to about 30 wt % of arginine.

The methods of improving insulin production and improving insulin secretion, as well as the nutritional compositions according to the present invention, are advantageous in that they improve insulin secretion and insulin production and thus may contribute to reduced β-cell deterioration and/or improved β-cell functionality. The methods and nutritional compositions of the invention can thus help delay or prevent the onset of diabetes, delay or prevent diabetes progression, and/or promote remission. These and additional objects and advantages of the invention will be more fully apparent in view of the following detailed description.

### BRIEF DESCRIPTION OF THE DRAWINGS

The embodiments set forth in the drawings are illustrative of certain aspects of the invention and exemplary in nature and are not intended to limit the invention defined by the claims, wherein:

FIG. 1 illustrates the effects of arginine (ARG), lysine (LYS), HMB, and the combination of arginine, lysine, and HMB (MIX) on glucose uptake in INS-1 cells, as described in Example 1.

FIG. 2 illustrates the effects of arginine (ARG), lysine (LYS), HMB, and the combination of arginine, lysine, and HMB (MIX) on the expression level of the main glucose transporter in pancreatic cells, GLUT2, in INS-1 cells, as described in Example 1.

FIG. 3 illustrates the effects of arginine (ARG), lysine (LYS), HMB, and the combination of arginine, lysine, and HMB (MIX) on the acute response of insulin secretion to extracellular glucose concentration in INS-1 cells, as described in Example 2.

FIG. 4 illustrates the effects of arginine (ARG), lysine (LYS), HMB, and the combination of arginine, lysine, and HMB (MIX) on insulin production in INS-1 cells, as described in Example 2.

FIG. 5 illustrates the effects of arginine (ARG), lysine (LYS), HMB, and the combination of arginine, lysine, and HMB (MIX) on intracellular insulin levels of INS-1 cells, as described in Example 2.

FIG. 6 illustrates the effects of arginine (ARG), lysine (LYS), HMB, and the combination of arginine, lysine, and HMB (MIX) on β-cell proliferation and viability, as described in Example 3.

### DETAILED DESCRIPTION

Specific embodiments of the invention are described herein. The invention can, however, be embodied in different forms and should not be construed as limited to the embodiments set forth herein. Rather, these embodiments are provided to illustrate more specific features of certain aspects of the invention to those skilled in the art.

The terminology as set forth herein is for description of the embodiments only and should not be construed as limiting the disclosure as a whole. All references to singular characteristics or limitations of the present disclosure shall include the corresponding plural characteristic or limitation, and vice versa, unless otherwise specified or clearly implied to the contrary by the context in which the reference is made. Unless otherwise specified, “a,” “an,” “the,” and “at least one” are used interchangeably. Furthermore, as used in the description and the appended claims, the singular forms “a,” “an,” and “the” are inclusive of their plural forms, unless the context clearly indicates otherwise.

To the extent that the term “includes” or “including” is used in the description or the claims, it is intended to be inclusive of additional elements or steps, in a manner similar to the term “comprising” as that term is interpreted when employed as a transitional word in a claim. Furthermore, to the extent that the term “or” is employed (e.g., A or B), it is intended to mean “A or B or both.” When the “only A or B but not both” is intended, then the term “only A or B but not both” is employed. Thus, use of the term “or” herein is the inclusive, and not the exclusive use. When the term “and” as well as “or” are used together, as in “A and/or B” this indicates A or B as well as A and B.

The methods and compositions described in the present disclosure can comprise, consist of, or consist essentially of any of the elements and steps as described herein.

All ranges and parameters, including but not limited to percentages, parts, and ratios disclosed herein are understood to encompass any and all sub-ranges subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, and 10) contained within the range.

Any combination of method or process steps as used herein can be performed in any order, unless otherwise specified or clearly implied to the contrary by the context in which the referenced combination is made.

All percentages are percentages by weight unless otherwise indicated.

The term “HMB” as used herein, unless otherwise specified, refers to beta-hydroxy-beta-methylbutyrate (also referred to as beta-hydroxyl-3-methyl butyric acid, beta-hydroxy isovaleric acid) and sources thereof. All weights, percentages, and concentrations as used herein to characterize HMB are based on the weight of HMB, regardless of the source, unless otherwise specified.

The term “calcium HMB” as used herein, unless otherwise specified, refers to the calcium salt of beta-hydroxy-beta-methylbutyrate (also referred to as beta-hydroxyl-3-methyl butyric acid, beta-hydroxy isovaleric acid, or HMB), which is most typically in a monohydrate form. All weights, percentages, and concentrations as used herein to characterize calcium HMB are based on the weight of calcium HMB monohydrate, unless otherwise specified.

The term “diabetes” as used herein, unless otherwise specified, refers to type 1 diabetes, type 2 diabetes, and gestational diabetes. Type 1 diabetes, also referred to as juvenile diabetes or insulin-dependent diabetes, is a chronic condition wherein the pancreas produces little or no insulin. It is considered an autoimmune condition, since the immune system mistakenly attacks and destroys the insulin-producing β-cells in the pancreas. Type 2 diabetes is a chronic condition that affects the way the body metabolizes sugar. In patients with type 2 diabetes, the body either resists the effects of insulin, resulting in reduced glucose uptake by cells, or does not produce enough insulin to maintain normal glucose levels. Type 2 diabetes often starts as insulin resistance, meaning that the body is not capable of using insulin efficiently. Gestational diabetes occurs only during pregnancy and is a result of insulin-blocking hormones that are produced during pregnancy. Patients diagnosed with gestational diabetes are at a higher risk of developing type 2 diabetes after pregnancy.

The term “nutritional powder” as used herein, unless otherwise specified, refers to nutritional powders that are generally flowable particulates and that are reconstitutable with an aqueous liquid, and which are suitable for oral administration to a human.

The term “nutritional liquid” as used herein, unless otherwise specified, refers to nutritional products in ready-to-drink liquid form and to nutritional liquids made by reconstituting the nutritional powders described herein prior to use.

The terms “nutritional product” and “nutritional composition” as used herein, unless otherwise specified, refer to nutritional liquids and nutritional powders, the latter of which may be reconstituted to form a nutritional liquid, and are suitable for oral consumption by a human.

Beta-hydroxy-beta-methylbutyrate (HMB) is a naturally occurring amino acid metabolite that is known for use in a variety of nutritional products and supplements. HMB is a metabolite of the essential amino acid leucine and has been shown to modulate protein turnover and inhibit proteolysis. Calcium HMB is a commonly used form of HMB when formulated in oral nutritional products, which products include tablets, capsules, reconstitutable powders, and nutritional liquids and emulsions.

While HMB is commonly used in nutritional products to help build or maintain healthy muscle in selected individuals, the present inventors have surprisingly discovered that HMB, in combination with arginine and lysine, improves insulin secretion and insulin production in pancreatic β-cells.

In one embodiment, a method of improving insulin production in a subject is provided. The method comprises administering a nutritional composition comprising lysine, arginine, and HMB to the subject. In another embodiment of the invention, a method of improving insulin secretion in a subject is provided. The method comprises administering a nutritional composition comprising lysine, arginine, and HMB to the subject. In specific embodiments of these methods, the subject is suffering from diabetes or prediabetes. In an additional embodiment of the methods of the invention, the method further comprises administering a sugar alcohol to the subject. Suitable sugar alcohols include, but are not limited to, mannitol, sorbitol, xylitol, lactitol, isomalt, maltitol, and myo-inositol. In a more specific embodiment, the sugar alcohol is myo-inositol.

In an additional embodiment of the invention, a nutritional composition comprising HMB, lysine and arginine is provided. In one embodiment, the nutritional composition comprises about 0.01 to about 15 wt %, or about 0.01 to about 10 wt % of HMB, about 0.03 to about 40 wt %, or about 0.03 to about 30 wt % of lysine, and about 0.02 to about 30 wt %, or about 0.02 to about 20 wt % of arginine. In specific embodiment of the nutritional compositions of the invention, the nutritional composition further comprises a sugar alcohol as described above. In a more specific embodiment, the sugar alcohol is myo-inositol.

In another embodiment of the nutritional compositions of the invention, the molar ratio of lysine to arginine in the nutritional composition is about 10:1 to about 1:1, or about 5:1 to about 1:1, or about 3:1 to about 1:1. In yet another specific embodiment, the molar ratio of a combination of lysine and arginine to HMB in the nutritional composition is about 15:1 to about 1:1, or about 10:1 to about 1:1, or about 5:1 to about 1:1, or about 3:1 to about 1:1.

Any source of HMB is suitable for use in the methods and nutritional compositions of the invention. Examples include HMB as the free acid, a salt, including an anhydrous salt or a hydrate salt, an ester, a lactone, or other product forms that otherwise provide a bioavailable form of HMB. In specific embodiments of the methods and compositions of the invention, the source of HMB is selected from the group consisting of alkali metal HMB, alkaline earth metal HMB, HMB free acid, HMB lactone and combinations thereof. In more specific embodiments, the source of HMB is selected from the group consisting of sodium HMB, potassium HMB, magnesium HMB, chromium HMB, calcium HMB and combinations thereof, or the HMB is calcium HMB monohydrate.

According to specific embodiments of the methods, lysine, arginine and HMB are administered orally. In a more specific embodiment, the lysine, arginine and HMB are provided in a nutritional composition and are administered orally.

The methods and nutritional compositions as described herein employ amounts of lysine, arginine, and HMB that are effective to improve insulin production and/or improve insulin secretion, and, more specifically, to achieve one or more of these effects to an extent greater than that achieved with lysine, arginine, or HMB alone.

In a specific embodiment, the nutritional composition is in the form of a powder. In another specific embodiment, the nutritional composition is in the form of a liquid.

In specific embodiments of the methods, the subject is administered about 1 to about 10 g, or about 2 to about 5 g of HMB per day.

In other specific embodiments of the methods, the subject is administered about 0.1 to about 30 g, or about 1 to about 10 g, or about 3 to about 6 g of lysine per day.

In further specific embodiments of the methods, the subject is administered about 0.1 to about 20 g, or about 1 to about 10 g, or about 1 to about 5 g of arginine per day.

Lysine and arginine can be added to the nutritional composition in either inherent or supplemented form. Inherent amino acids are those provided by dietary proteins, whereas supplemented amino acids are the free amino acids in the L- or D- configuration. In specific embodiments of the invention, the nutritional composition employs supplemental lysine and/or arginine. In more specific embodiments of the invention, the nutritional composition employs lysine and/or arginine in the L- form.

In further specific embodiments of the invention, the nutritional composition comprises about 0.01 to about 10 wt %, about 0.01 to about 8 wt %, about 0.01 to about 5.0 wt %, 0.1 to about 10 wt %, about 0.1 to about 8 wt %, about 0.1 to about 5.0 wt %, about 0.2 to about 5.0 wt %, about 0.3 to about 3 wt %, about 0.3 to about 2 wt %, about 0.3 to about 1.5 wt %, about 0.3 to about 1.0 wt %, about 0.3 to about 0.6 wt %, or about 0.4 to about 1.5 wt % of HMB, based on the weight of the nutritional composition.

In another specific embodiment, the nutritional composition comprises about 0.03 to about 40 wt %, or about 0.03 to about 30 wt %, or about 0.03 to about 20 wt % of lysine, based on the weight of the nutritional composition. In a more specific embodiment, the nutritional composition comprises about 0.1 to about 10 wt % of lysine, based on the weight of the nutritional composition.

In another specific embodiment of the invention, the nutritional composition comprises about 0.02 to about 30 wt %, or about 0.02 to about 20 wt %, or about 0.02 to about 10 wt % of arginine, based on the weight of the nutritional composition. In a more specific embodiment, the nutritional composition comprises about 0.05 to about 5 wt % of arginine, based on the weight of the nutritional composition.

In other specific embodiments of the invention, the nutritional composition further comprises protein, carbohydrate, and/or fat, in any amounts as desired. A wide variety of sources and types of protein, carbohydrate, and fat can be used in embodiments of nutritional compositions described herein. In a specific embodiment, the nutritional composition includes protein, carbohydrate and fat.

In further specific embodiments, the protein in the nutritional composition comprises whey protein concentrate, whey protein isolate, whey protein hydrolysate, milk protein concentrate, milk protein isolate, milk protein hydrolysate, organic milk protein concentrate, soy protein concentrate, soy protein isolate, soy protein hydrolysate, pea protein concentrate, pea protein isolate, pea protein hydrolysate, acid casein, sodium caseinate, calcium caseinate, potassium caseinate, casein hydrolysate, nonfat dry milk, condensed skim milk, collagen protein, collagen protein isolate, L-Carnitine, taurine, lutein, rice protein concentrate, rice protein isolate, rice protein hydrolysate, fava bean protein concentrate, fava bean protein isolate, fava bean protein hydrolysate, meat protein, potato protein, chickpea protein, canola protein, mung protein, quinoa protein, amaranth protein, chia protein, hemp protein, flax seed protein, earthworm protein, insect protein, or combinations of two or more thereof.

In specific embodiments, the nutritional composition may comprise protein in an amount about 1 wt % to about 30 wt % of the nutritional composition. More specifically, the protein may be present in an amount about 1 wt % to about 25 wt % of the nutritional composition, including about 1 wt % to about 20 wt %, about 2 wt % to about 20 wt %, about 1 wt % to about 15 wt %, about 1 wt % to about 10 wt %, about 5 wt % to about 10 wt %, about 10 wt % to about 25 wt %, or about 10 wt % to about 20 wt % of the nutritional composition. Even more specifically, the protein comprises about 1 wt % to about 5 wt % of the nutritional composition, or about 20 wt % to about 30 wt % of the nutritional composition.

In other specific embodiments, the carbohydrate in the nutritional composition comprises fiber, human milk oligosaccharides (HMOs), maltodextrin, corn maltodextrin, corn syrup, organic corn maltodextrin, corn syrup, corn syrup solids, sucralose, cellulose gel, cellulose gum, gellan gum, carrageenan, fructooligosaccharides (FOS), inositol, hydrolyzed starch, glucose polymers, rice-derived carbohydrates, sucrose, glucose, lactose, honey, sugar alcohols, isomaltulose, sucromalt, pullulan, potato starch, galactooligosaccharides, oat fiber, soy fiber, corn fiber, gum arabic, sodium carboxymethylcellulose, methylcellulose, guar gum, locust bean gum, konjac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, psyllium, inulin, and combinations of two or more thereof. In further specific embodiments, the carbohydrate in the nutritional composition comprises a combination of two or more carbohydrates, wherein the carbohydrates have varying rates of absorption. In specific embodiments, the carbohydrate that may be used in the nutritional composition of the invention comprises isomaltulose, sucromalt, resistant maltodextrin (e.g., Fibersol or Nutriose), FOS, inulin, oat fiber, soy fiber, or a combination of two or more thereof.

In specific embodiments, the nutritional composition may comprise carbohydrate in an amount about 0.5 wt % to about 75 wt % of the nutritional composition. More specifically, the carbohydrate may be present in an amount about 1 wt % to about 70 wt % of the nutritional composition, including about 5 wt % to about 70 wt %, about 5 wt % to about 65 wt %, about 5 wt % to about 50 wt %, about 5 wt % to about 40 wt %, about 5 wt % to about 30 wt %, about 5 wt % to about 25 wt %, about 10 wt % to about 65 wt %, about 20 wt % to about 65 wt %, about 30 wt % to about 65 wt %, about 40 wt % to about 65 wt %, about 40 wt % to about 70 wt %, or about 15 wt % to about 25 wt %, of the nutritional composition.

The terms “fat” and “oil” as used herein, unless otherwise specified, are used interchangeably to refer to lipid materials derived or processed from plants or animals. These terms also include synthetic lipid materials so long as such synthetic materials are suitable for oral administration to humans.

In further specific embodiments, the fat comprises coconut oil, fractionated coconut oil, soy oil, soy lecithin, corn oil, safflower oil sunflower oil, palm olein, canola oil monoglycerides, lecithin, canola oil, medium chain triglycerides, one or more fatty acids such as linoleic acid, alpha-linolenic acid, fractionated coconut oil, soy oil, corn oil, olive oil, safflower oil, medium chain triglyceride oil (MCT oil), high gamma linolenic (GLA) safflower oil, palm oil, palm kernel oil, marine oil, fish oil, algal oil, borage oil, cottonseed oil, fungal oil, interesterified oil, transesterified oil, structured lipids, omega-3 fatty acid, or combinations of two or more thereof. In a specific embodiment, the omega-3 fatty acid is selected from the group consisting of eicosapentaenoic acid, docosahexaenoic acid, arachidonic acid, and alpha-linolenic acid, and combinations of two or more thereof.

In specific embodiments, the nutritional composition may comprise fat in an amount of about 0.5 wt % to about 30 wt % of the nutritional composition. More specifically, the fat may be present in an amount about 0.5 wt % to about 10 wt %, about 1 wt % to about 30 wt % of the nutritional composition, including about 1 wt % to about 20 wt %, about 1 wt % to about 15 wt %, about 1 wt % to about 10 wt %, about 1 wt % to about 5 wt %, about 3 wt % to about 30 wt %, about 5 wt % to about 30 wt %, about 5 wt % to about 25 wt %, about 5 wt % to about 20 wt %, about 5 wt % to about 10 wt %, or about 10 wt % to about 20 wt % of the nutritional composition.

The concentration and relative amounts of the sources of protein, carbohydrate, and fat in the exemplary nutritional compositions can vary considerably depending upon, for example, the specific dietary needs of the intended user. In a specific embodiment, the nutritional composition comprises a source of protein in an amount about 2 wt % to about 20 wt %, a source of carbohydrate in an amount about 5 wt % to about 30 wt %, and a source of fat in an amount about 0.5 wt % to about 10 wt %, based on the weight of the nutritional composition, and, more specifically, such composition is in liquid form. In another specific embodiment, the nutritional composition comprises a source of protein in an amount about 10 wt % to about 25 wt %, a source of carbohydrate in an amount about 40 wt % to about 70 wt %, and a source of fat in an amount of about 5 wt % to about 20 wt %, based on the weight of the nutritional composition, and, more specifically, such composition is in powder form.

In one embodiment, the nutritional composition is a nutritional liquid composition and comprises about 1 to about 15 wt % of protein, about 0.5 to about 10 wt % fat, and about 5 to about 30 wt % carbohydrate, based on the weight of the nutritional composition.

In another embodiment, the nutritional composition is a nutritional powder composition and comprises about 10 to about 30 wt % of protein, about 5 to about 15 wt % fat, and about 30 wt % to about 65 wt % carbohydrate, based on the weight of the nutritional composition.

In a specific embodiment, the nutritional composition comprises at least one protein comprising milk protein concentrate and/or soy protein isolate, at least one fat comprising canola oil, corn oil, coconut oil and/or marine oil, and at least one carbohydrate comprising maltodextrin, resistant maltodextrin, sucrose, and/or short-chain fructooligosaccharide.

The nutritional composition may also comprise one or more components to modify the physical, chemical, aesthetic, or processing characteristics of the nutritional composition or serve as additional nutritional components. Non-limiting examples of additional components include preservatives, emulsifying agents (e.g., lecithin), buffers, sweeteners including artificial sweeteners (e.g., saccharine, aspartame, acesulfame K, sucralose), colorants, flavorants, thickening agents, stabilizers, and so forth.

In specific embodiments, the nutritional composition has a neutral pH, i.e., a pH of about 6 to 8 or, more specifically, about 6 to 7.5. In more specific embodiments, the nutritional composition has a pH of about 6.5 to 7.2 or, more specifically, about 6.8 to 7.1.

The nutritional composition may be formed using any techniques known in the art. In one embodiment, the nutritional composition may be formed by (a) preparing an aqueous solution comprising protein and carbohydrate; (b) preparing an oil blend comprising fat and oil-soluble components; and (c) mixing together the aqueous solution and the oil blend to form an emulsified liquid nutritional composition. The HMB, lysine, and arginine can be added at any point in the formation of the nutritional composition.

As indicated above, the nutritional composition can be administered in the form of a powder or in the form of a liquid.

When the nutritional composition is a powder, for example, a serving size is about 40 g to about 60 g, such as 45 g, or 48.6 g, or 50 g, to be administered as a powder or to be reconstituted in about 1 ml to about 500 ml of liquid.

When the nutritional composition is in the form of a liquid, for example, reconstituted from a powder or manufactured as a ready-to-drink product, a serving ranges about 1 ml to about 500 ml, including about 110 ml to about 500 ml, about 110 ml to about 417 ml, about 120 ml to about 500 ml, about 120 ml to about 417 ml, about 177 ml to about 417 ml, about 207 ml to about 296 ml, about 230 m to about 245 ml, about 110 ml to about 237 ml, about 120 ml to about 245 ml, about 110 ml to about 150 ml, and about 120 ml to about 150 ml. In specific embodiments, the serving is about 1 ml, or about 100 ml, or about 225 ml, or about 237 ml, or about 500 ml.

In specific embodiments, the nutritional composition comprising HMB, lysine and arginine is administered to a subject once or multiple times daily or weekly. In specific embodiments, the nutritional composition is administered to the subject about 1 to about 6 times per day or per week, or about 1 to about 5 times per day or per week, or about 1 to about 4 times per day or per week, or about 1 to about 3 times per day or per week. In specific embodiments, the nutritional composition is administered once or twice daily for a period of at least one week, at least two weeks, at least three weeks, or at least four weeks.

In another embodiment of the invention, the nutritional composition comprises protein, carbohydrate, fat, and one or more nutrients selected from the group consisting of vitamins and minerals. Specific embodiments of the nutritional composition may comprise vitamins and/or related nutrients, non-limiting examples of which include vitamin A, vitamin B12, vitamin C, vitamin D, vitamin E, vitamin K, thiamine, riboflavin, pyridoxine, niacin, folic acid, pantothenic acid, biotin, choline, inositol, and/or salts and derivatives thereof, and combinations thereof.

Specific embodiments of the nutritional composition comprise minerals, non-limiting examples of which include calcium, phosphorus, magnesium, zinc, manganese, sodium, potassium, molybdenum, chromium, iron, copper, and/or chloride, and combinations thereof.

The following Examples demonstrate aspects of the inventive methods and are provided solely for the purpose of illustration. The Examples are not to be construed as limiting of the general inventive concepts, as many variations thereof are possible without departing from the spirit and scope of the general inventive concepts.

### Examples

### Example 1: Effects of HMB, Lysine, and Arginine on Glucose Uptake in the Rat Insulinoma Cell Line (INS-1)

This example describes the use of the rat insulinoma cell line (INS-1), which is capable of insulin release in response to glucose stimulation, to detect changes to glucose metabolism and insulin secretion, as well as the expression level of the pancreatic glucose transporter GLUT2.

The INS-1 cells were maintained in RPMI 1640 medium with 11.1 mmol/L D-glucose supplemented with 10% fetal bovine serum, 100 units/mL penicillin, 100 μg/mL streptomycin, 10 mmol/L HEPES, 2 mmol/L L-glutamine, 1 mmol/L sodium pyruvate, and 50 μmol/L β-mercaptoethanol at 37° C./5% CO₂ in a humidified atmosphere.

The cells were incubated with different effectors overnight. The following cell groups were employed:
(i) Control: INS-1 cells without ingredients.
(ii) ARG: INS-1 cells incubated in the presence of 5 mM of arginine.
(iii) LYS: INS-1 cells incubated in the presence of 10 mM of lysine.
(iv) HMB: INS-1 cells incubated in the presence of 25 μM of HMB-free acid.
(v) MIX: INS-1 cells incubated in the presence of the combination of 5 mM of arginine, 10 mM of lysine, and 25 μM of HMB.

2-deoxy-[3H]d-glucose (2-DG) (10 μmol/L) uptake was measured over a 10 minute period under conditions in which the uptake was linear. The uptake of 2-DG was terminated after 10 minutes by rapidly aspirating off the radioactive incubation medium and washing the cells three times in ice-cold phosphate-buffered saline. The radioactivity associated with the cells was determined by cell lysis in 0.5 N NaOH with neutralization by the addition of 0.5 N HCl, followed by liquid scintillation. Aliquots from each well were used to determine the protein concentration using the bicinchoninic acid (BCA) Protein assay.

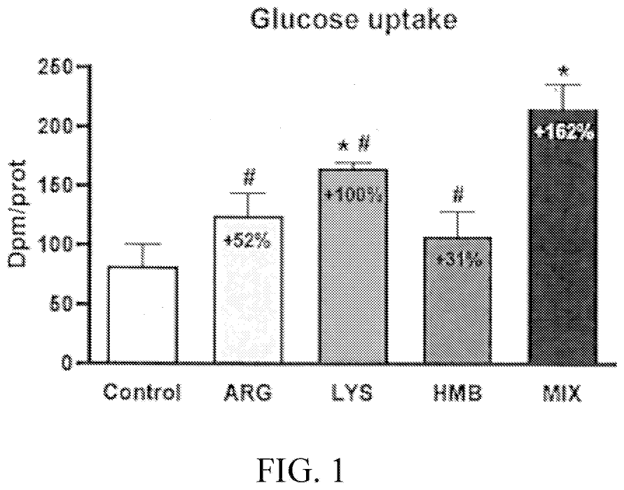

As illustrated in FIG. 1, incubating INS-1 cells with the individual amino acids, lysine and arginine, increased glucose uptake. Lysine (10 mM) increased glucose uptake by about 100% compared to the control, arginine (5 mM) increased glucose uptake about 52% compared to the control, and HMB increase glucose uptake by about 31% compared to the control. However, when the cells were incubated with the combination of lysine (10 mM), arginine (5 mM), and HMB (25 μM), there was a significant increase in glucose uptake. As shown in FIG. 1, cells incubated with the combination of both amino acids and HMB showed about a 162% increase in glucose uptake compared to the control, and about a 31% increase in glucose uptake compared to incubation with lysine alone. These results demonstrate that the combination of lysine, arginine, and HMB exhibits a surprising synergistic effect on glucose uptake in β-cells.

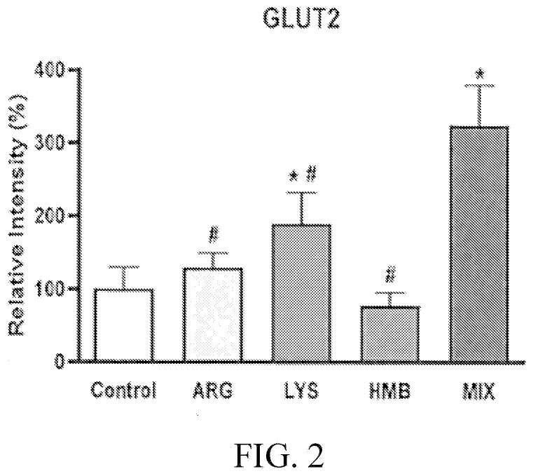

FIG. 2 illustrates the effects of lysine (10 mM), arginine (5 mM), HMB (25 μM), and their combination on the expression level of the main glucose transporter in pancreatic cells, GLUT2. The incubation with lysine (10 mM) alone resulted in about a 90% increase in the relative intensity of the expression level of GLUT2 compared to control, and the incubation with arginine (5 mM) resulted in about a 30% increase in the relative intensity of the expression level of GLUT2 compared to control. However, while the incubation with HMB (25 μM) alone resulted in about a 23% decrease in the relative intensity of the expression level of GLUT2 compared to control, the combination of lysine (10 mM), arginine (5 mM), and HMB (25 μM) exhibited a surprising synergistic effect on glucose uptake. Incubating the cells with the combination of lysine (10 mM), arginine (5 mM), and HMB (25 μM) resulted in about a 220% increase in the relative intensity of the expression level of GLUT2 compared to the control.

These results show that the combination of arginine, lysine, and HMB exhibits a synergistic effect on glucose uptake in INS-1 cells.

### Example 2: Effects of HMB, Lysine, and Arginine on Insulin Secretion and Insulin Production in the Rat Insulinoma Cell Line (INS-1)

This example describes the use of the rat insulinoma cell line to detect acute insulin secretion, as well as insulin production.

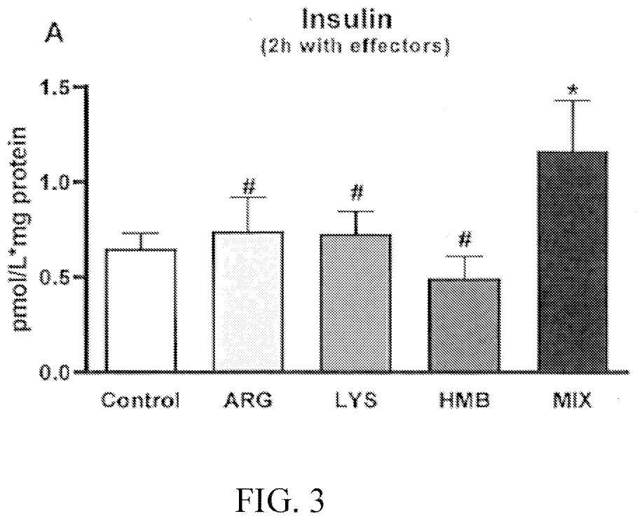

Acute insulin secretion was determined by incubating INS-1 cells in the presence of the following effectors: glucose (10 mM), and the effectors arginine (5 mM), lysine (10 mM), HMB (25 μM), and a combination of arginine (5 mM), lysine (10 mM), and HMB (25 μM) for 2 hours. INS-1 cells without effectors were used as a control. Insulin was detected using the Rat Insulin Enzyme Immunoassay (RIEI) Kit (Mercodia, Sweden) according to the manufacturer's instructions. As illustrated in FIG. 3, the acute response of insulin secretion to extracellular glucose concentration in the individual presence of the arginine, lysine, and HMB did not show a significant difference with respect to the control. However, the combination of arginine, lysine, and HMB significantly increased insulin secretion by approximately 79% compared to the control. This result demonstrates that the combination of arginine, lysine, and HMB exhibits a surprising synergistic effect on insulin secretion in β-cells.

Insulin production by β-cells was evaluated by incubating INS-1 cells in the presence of the following effectors: arginine (5 mM), lysine (10 mM), HMB (25 μM), and a combination of arginine (5 mM), lysine (10 mM), and HMB (25 μM). INS-1 cells without effectors were again used as a control. The cells were incubated with each of the above mentioned effectors for 24 hours. Following the initial 24 hour incubation period with the effectors, the same INS-1 cells were incubated with glucose (10 mM) for 2 hours. Insulin was again detected using the RIEI Kit according to the manufacturer's instructions.

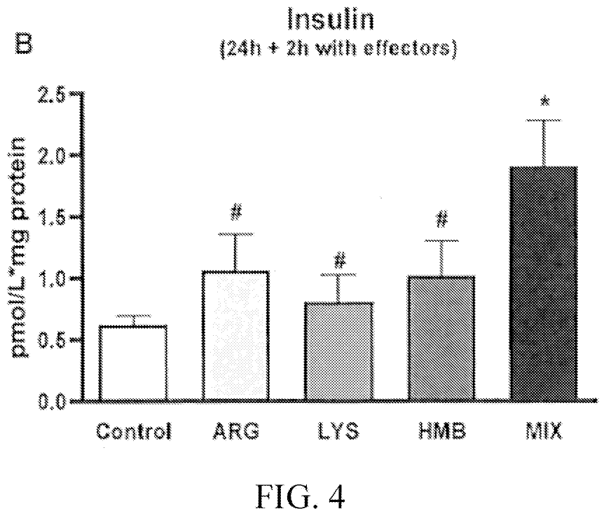

As illustrated in FIG. 4, insulin production in cells incubated with the individual effectors arginine (5 mM), lysine (10 mM), and HMB (25 μM) rose slightly compared to the control, while, surprisingly, there was a 207% in insulin production observed in INS-1 cells incubated with the combination of arginine (5 mM), lysine (10 mM), and HMB (25 μM). These results indicate that the combination of arginine (5 mM), lysine (10 mM), and HMB (25 μM) exhibits a surprising synergistic effect on stimulating the production of insulin in pancreatic β-cells.

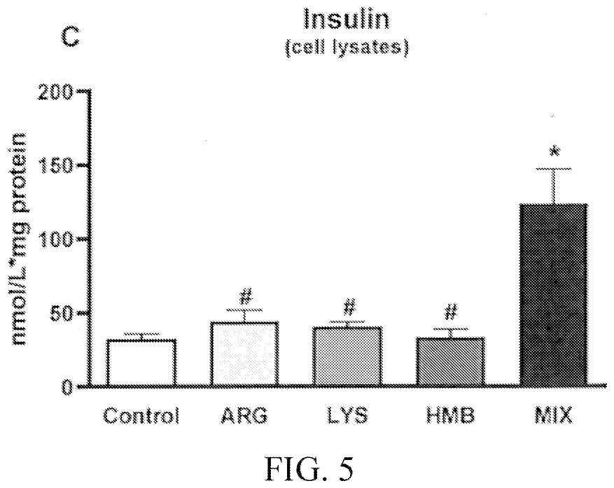

The above mentioned effects of increased insulin secretion and production in INS-1 cells incubated with a combination of arginine (5 mM), lysine (10 mM), and HMB (25 μM) was confirmed by incubating the INS-1 cells for 24 hours with each of the above mentioned effectors for 24 hours and subsequently lysing the cells. Protein concentration was measured using the BCA assay. As illustrated in FIG. 5, the intracellular insulin levels also indicated the synergistic effect observed with the combination of arginine, lysine, and HMB by increasing the insulin production significantly, as compared to both control and the individual effectors.

### Example 3: Effects of HMB, Lysine, and Arginine on the Preservation of β-Cell Mass in the Rat Insulinoma Cell Line (INS-1)

This example describes the use of the rat insulinoma cell line (INS-1) to detect the effect of the combination of lysine, arginine, and HMB on the functional characteristics of β-cells in the presence of free fatty acids (e.g., palmitic acid) using standardized protocols for viability. In type 2 diabetes patients, chronic increases of plasma free fatty acids concentrations result in disturbances in lipid metabolism regulation, which contribute to decreased beta-cell function and viability (lipotoxicity). The INS-1 cells were maintained in RPMI 1640 medium with 11.1 mmol/L D-glucose supplemented with 10% fetal bovine serum, 100 units/mL penicillin, 100 μg/mL streptomycin, 10 mmol/L HEPES, 2 mmol/L L-glutamine, 1 mmol/L sodium pyruvate, and 50 μmol/L β-mercaptoethanol at 37° C./5% CO₂ in a humidified atmosphere.

The pre-confluent pancreatic cells were left either untreated (Control) or treated with palmitate at a concentration of 250 μM for 24 hours in complete medium (Palmitate). Three hours before incubation with palmitate, four different effectors were added: arginine (5 mM), lysine (10 mM), HMB (25 μM), and the combination of arginine (5 mM), lysine (10 mM), and HMB (25 μM).

The cell groups employed in the study were the following:
(i) Control: INS-1 cells without ingredients.
(ii) Palmitate: INS-1 cells incubated in the presence of 250 μM palmitate.
(iii) Palmitate+ARG: INS-1 cells incubated in the presence of 250 μM palmitate and 5 mM of arginine.
(iv) Palmitate+LYS: INS-1 cells incubated in the presence of 250 μM palmitate and 10 mM of lysine.
(v) Palmitate+HMB: INS-1 cells incubated in the presence of 250 μM palmitate and 25 μM of HMB-free acid.
(vi) Palmitate+MIX: INS-1 cells incubated in the presence of 250 μM palmitate and the combination of 5 mM of arginine, 10 mM of lysine, and 25 μM of HMB.

A cell viability assay was performed by the indirect measurement of cell metabolic activity using MTT (3-(4,5-dimethylthiazol-2-yl)-2,5-diphenyltetrazolium bromide). After 24 hours of incubation with the above effectors, the MTT solution (0.5 mg/ml) was added for 30 minutes. The supernatant was removed, 100 μl MTT solvent was added to each well, and the culture plate was shaken for 10 minutes before the Optical Density (OD) values were recorded at 570 nm.

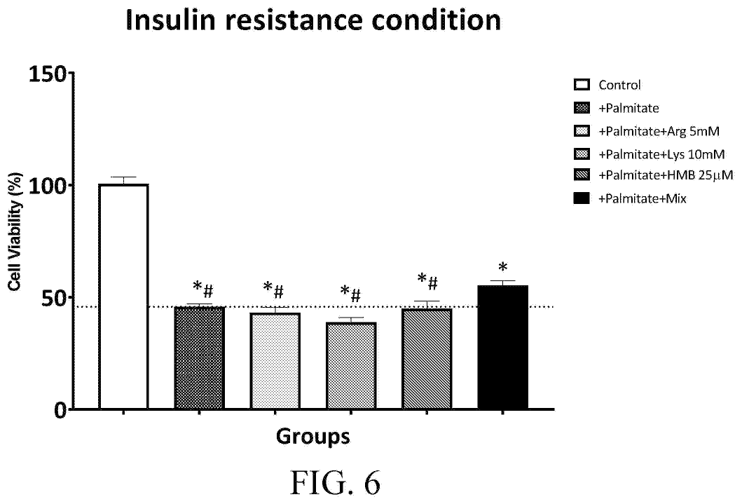

As illustrated in FIG. 6, cell viability at 24 hours was negatively affected by palmitate incubation. Data are presented as media ±SEM. A p value <0.05 was considered significant versus (*) Control group, or (#) Mix group. Incubation with the individual amino acids and HMB did not demonstrate a positive effect on the exposure of the cells to palmitate. In fact, incubation with arginine, lysine, and HMB individually reduced cell viability as compared to treatment with palmitate alone. However, the combination of lysine, arginine, and HMB (i.e., Mix) prevented the negative effect of palmitate on cell viability, showing significantly higher cell proliferation in comparison with the rest of the palmitate-treated groups. This increase was around 20% higher than cells incubated with palmitate alone. These results show that the combination of lysine, arginine, and HMB exhibits a synergistic effect on improving β-cell mass and thus functionality.

The results of these Examples demonstrate the surprising synergistic effects that a combination of lysine, arginine, and HMB have on enhancing insulin production and secretion in pancreatic β-cells. These results indicate that administration of the combination of lysine, arginine, and HMB improves β-cell functionality, which is particularly relevant for the treatment of diabetes and prediabetes, as well as for preventing the progression of diabetes and its related morbidities.

While the present invention has been illustrated by the description of embodiments thereof, and while the embodiments have been described in considerable detail, such descriptions are not intended to restrict or in any way limit the scope of the appended claims to such detail. Additional advantages and modifications will readily appear to those skilled in the art. Therefore, the invention, in its broader aspects, is not limited to the specific details, the representative compositions and methods, or illustrative examples shown and described. Accordingly, departures may be made from such details without departing from the spirit or scope of the general inventive concept.

1. A method of improving insulin production and/or improving insulin secretion in a subject in need thereof, comprising: administering a nutritional composition comprising lysine, arginine, and beta-hydroxy-beta-methylbutyrate (HMB) to the subject.

2. (canceled)

3. The method of claim 1, wherein the subject is suffering from diabetes or prediabetes.

4. The method of claim 1, wherein the method further comprises administering a sugar alcohol, or administering myo-inositol, to the subject.

5. The method of claim 1, wherein the HMB is selected from the group consisting of alkali metal HMB, alkaline earth metal HMB, HMB free acid, HMB lactone and combinations thereof, or the HMB is selected from the group consisting of sodium HMB, potassium HMB, magnesium HMB, chromium HMB, calcium HMB and combinations thereof, or the HMB is calcium HMB monohydrate.

6. The method of claim 1, wherein the molar ratio of lysine to arginine in the nutritional composition is about 10:1 to about 1:1, or about 5:1 to about 1:1, or about 3:1 to about 1:1, and wherein the molar ratio of a combination of lysine and arginine to HMB in the nutritional composition is about 15:1 to about 1:1, or about 10:1 to about 1:1, or about 5:1 to about 1:1, or about 3:1 to about 1:1.

7. The method of claim 1, wherein the nutritional composition is administered to the subject orally.

8. (canceled)

9. (canceled)

10. The method of claim 1, wherein the subject is administered about 1 to about 10 g, or about 2 to about 5 g of HMB per day; and/or about 0.1 to about 30 g, about 1 to about 10 g, or about 4 to about 5 g of lysine per day; and/or about 0.1 to about 20 g, about 1 to about 10 g, or about 2 to about 3 g of arginine per day.

11. The method of claim 1, wherein the nutritional composition further comprises protein, carbohydrate, and/or fat.

12. The method of claim 11, wherein the protein comprises whey protein concentrate, whey protein isolate, whey protein hydrolysate, milk protein concentrate, milk protein isolate, milk protein hydrolysate, organic milk protein concentrate, soy protein concentrate, soy protein isolate, soy protein hydrolysate, pea protein concentrate, pea protein isolate, pea protein hydrolysate, acid casein, sodium caseinate, calcium caseinate, potassium caseinate, casein hydrolysate, nonfat dry milk, condensed skim milk, collagen protein, collagen protein isolate, L-Carnitine, taurine, lutein, rice protein concentrate, rice protein isolate, rice protein hydrolysate, fava bean protein concentrate, fava bean protein isolate, fava bean protein hydrolysate, meat protein, potato protein, chickpea protein, canola protein, mung protein, quinoa protein, amaranth protein, chia protein, hemp protein, flax seed protein, earthworm protein, insect protein, or combinations of two or more thereof.

13. The method of claim 11, wherein the carbohydrate comprises fiber, human milk oligosaccharides (HMOs), maltodextrin, resistant maltodextrin, corn maltodextrin, organic corn maltodextrin, corn syrup, corn syrup solids, sucralose, cellulose gel, cellulose gum, gellan gum, carrageenan, fructooligosaccharides (FOS), inositol, hydrolyzed starch, glucose polymers, rice-derived carbohydrates, sucrose, glucose, lactose, honey, isomaltulose, sucromalt, pullulan, potato starch, galactooligosaccharides, oat fiber, soy fiber, corn fiber, gum arabic, sodium carboxymethylcellulose, methylcellulose, guar gum, locust bean gum, konj ac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, psyllium, inulin, and combinations of two or more thereof.

14. The method of claim 13, wherein the carbohydrate comprises isomaltulose, sucromalt, maltodextrin, resistant maltodextrin, FOS, inulin, oat fiber, soy fiber, or a combination of two or more thereof.

15. The method of claim 11, wherein the fat comprises coconut oil, fractionated coconut oil, soy oil, soy lecithin, corn oil, safflower oil, sunflower oil, palm olein, canola oil monoglycerides, lecithin, canola oil, medium chain triglycerides, linoleic acid, alpha-linolenic acid, olive oil, medium chain triglyceride oil (MCT oil), high gamma linolenic (GLA) safflower oil, palm oil, palm kernel oil, marine oil, fish oil, algal oil, borage oil, cottonseed oil, fungal oil, interesterified oil, transesterified oil, structured lipids, omega-3 fatty acid, or combinations of two or more thereof.

16. (canceled)

17. A nutritional composition comprising:
about 0.01 to about 15 wt % HMB;
about 0.03 to about 40 wt % lysine; and
about 0.02 to about 30 wt % arginine, all based on the weight of the nutritional composition.

18. The nutritional composition of claim 17, wherein the nutritional composition comprises: about 0.01 to about 10 wt %, about 0.01 to about 8 wt %, about 0.01 to about 5.0 wt %, about 0.1 to about 10 wt %, about 0.1 to about 8 wt %, about 0.1 to about 5.0 wt %, about 0.2 to about 5.0 wt %, about 0.3 to about 3 wt %, about 0.3 to about 2 wt %, about 0.3 to about 1.5 wt %, about 0.3 to about 1.0 wt %, about 0.3 to about 0.6 wt %, or about 0.4 to about 1.5 wt % of HMB; about 0.03 to about 30 wt %, about 0.03 to about 20 wt %, or about 0.1 to about 10 wt % of lysine; and about 0.02 to about 20 wt %, about 0.02 to about 10 wt %, or about 0.05 to about 5 wt % of arginine, all based on the weight of the nutritional composition.

19. The nutritional composition of claim 17, wherein the nutritional composition further comprises a sugar alcohol, or further comprises myo-inositol.

20. The nutritional composition of claim 17, wherein the nutritional composition further comprises protein, carbohydrate, and/or fat.

21. The nutritional composition of claim 20, wherein the protein comprises whey protein concentrate, whey protein isolate, whey protein hydrolysate, milk protein concentrate, milk protein isolate, milk protein hydrolysate, organic milk protein concentrate, soy protein concentrate, soy protein isolate, soy protein hydrolysate, pea protein concentrate, pea protein isolate, pea protein hydrolysate, acid casein, sodium caseinate, calcium caseinate, potassium caseinate, casein hydrolysate, nonfat dry milk, condensed skim milk, collagen protein, collagen protein isolate, L-Carnitine, L-Lysine, taurine, lutein, rice protein concentrate, rice protein isolate, rice protein hydrolysate, fava bean protein concentrate, fava bean protein isolate, fava bean protein hydrolysate, collagen protein, collagen protein isolate, meat protein, potato protein, chickpea protein, canola protein, mung protein, quinoa protein, amaranth protein, chia protein, hemp protein, flax seed protein, earthworm protein, insect protein, or combinations of two or more thereof.

22. The nutritional composition of claim 20, wherein the carbohydrate comprises fiber, human milk oligosaccharides (HMOs), maltodextrin, resistant maltodextrin, corn maltodextrin, organic corn maltodextrin, corn syrup, corn syrup solids, sucralose, cellulose gel, cellulose gum, gellan gum, carrageenan, fructooligosaccharides, inositol, hydrolyzed starch, glucose polymers, rice-derived carbohydrates, sucrose, glucose, lactose, honey, isomaltulose, sucromalt, pullulan, potato starch, galactooligosaccharides, oat fiber, soy fiber, corn fiber, gum arabic, sodium carboxymethylcellulose, methylcellulose, guar gum, locust bean gum, konj ac flour, hydroxypropyl methylcellulose, tragacanth gum, karaya gum, gum acacia, chitosan, arabinoglactins, glucomannan, xanthan gum, alginate, pectin, low methoxy pectin, high methoxy pectin, cereal beta-glucans, psyllium, inulin, and combinations of two or more thereof.

23. The nutritional composition of claim 22, wherein the carbohydrate comprises isomaltulose, sucromalt, maltodextrin, resistant maltodextrin, FOS, inulin, oat fiber, soy fiber, or a combination of two or more thereof

24. The nutritional composition of claim 20, wherein the fat comprises coconut oil, fractionated coconut oil, soy oil, soy lecithin, corn oil, safflower oil, sunflower oil, palm olein, canola oil monoglycerides, lecithin, canola oil, medium chain triglycerides, one or more fatty acids such as linoleic acid, alpha-linolenic acid, olive oil, medium chain triglyceride oil (MCT oil), high gamma linolenic (GLA) safflower oil, palm oil, palm kernel oil, marine oils, fish oils, algal oils, borage oil, cottonseed oil, fungal oils, interesterified oils, transesterified oils, structured lipids, omega-3 fatty acid, or combinations of two or more thereof.

25. (canceled)

26. The nutritional composition of claim 17, wherein the nutritional composition comprises about 1 to about 15 wt % of protein, about 0.5 to about 10 wt % fat, and about 1 to about 20 wt % carbohydrate, based on the weight of the nutritional composition.
